(19) United States
(12) Patent Application Publication
(10) Pub. No.: US 2022/0104758 A1
(43) Pub. Date: Apr. 7, 2022

(54) Device And Method For Sleep Monitoring

(71) Applicant: Nitto Denko Corporation, Osaka (JP)

(72) Inventors: Khine Cho Cho Thein, Singapore (SG); William Tan, Singapore (SG); Kittipong Kasamsook, Singapore (SG)

(21) Appl. No.: 17/451,938

(22) Filed: Oct. 22, 2021

Related U.S. Application Data
(63) Continuation of application No. 15/540,670, filed on Jun. 29, 2017.

(51) Int. Cl. A61B 5/00; A61B 5/024; A61B 5/11

(52) U.S. Cl. CPC A61B 5/4812; A61B 5/11; A61B 5/02416; A61B 5/02405

(57) ABSTRACT

A device and method for sleep monitoring, in particular to a device and method for determining time-to-sleep and wake periods during sleep and to a device and method for determining rapid eye movement (REM) sleep and non REM (NREM) sleep. The method for determining time-to-sleep and wake periods during sleep comprising the steps of obtaining motion data representative of motion of a user; detecting the time-to-sleep from the motion data based on a first time-above-threshold (TAT) threshold and a first proportional integration method (PIM) threshold; and detecting the wake periods during sleep from the motion data based on a second TAT threshold and a second PIM threshold.

### RELATED APPLICATION

This application is a continuation of U.S. application Ser. No. 15/540,670, filed Jun. 29, 2017, which is the U.S. National Stage of International Application No. PCT/SG2014/000624, filed on Dec. 30, 2014, published in English. The entire teachings of the above application(s) are incorporated herein by reference

### FIELD OF INVENTION

The present invention relates broadly to a device and method for sleep monitoring, in particular to a device and method for determining time-to-sleep and wake periods during sleep and to a device and method for determining rapid eye movement (REM) sleep and non REM (NREM) sleep.

### BACKGROUND

Having a good sleep at night is a key to perform the best during the day and to keep the fitness and well-being.

Many research studies show that there is a major link between sleep problems and a variety of serious health conditions including depression, heart disease, obesity and shorter life expectancy. Only loss of one hour of sleep in several nights can cause significantly negative effect on performance, learning skill, mood and safety. Long night sleepers who sleep more than 9 hrs or more also show risk of coronary heart disease and risk of stroke.

A personal device for sleep monitoring is thus desirable.

To track sleep condition, important parameters as referred to herein are Time-to-sleep, Total time-in-bed, Total sleep time, Time awake during sleep, Sleep efficiency and sleep quality (architecture/stages).

Time-to-sleep is also called Sleep Latency or Sleep Onset. Normal people without significant sleep deprivation typically take more than 20 minutes to fall asleep. Time-to-sleep is also correlated to sleep deprivation by referring to the MSLT (Multiple Sleep Latency Test) table shown in Table 1 below. MSLT provides a sleepiness of a subject and the severity of their sleep debt from the time taken to fall asleep.

TABLE 1
MSLT Scores

| Minutes | Sleepiness |
|---|---|
| 0-5 | Severe |
| 5-10 | Troublesome |
| 10-15 | Manageable |
| 15-20 | Excellent |

Total time-in-bed is the recorded time that the user has spent in-bed in total when they have entered and exited the sleep monitoring mode.

Total sleep time is the total sleep time recorded, which is the difference between the total time-in-bed and the time awake during sleep.

Time awake during sleep are the periods of wakefulness/restlessness identified during sleep and the record of the number of times awake and their duration.

Sleep efficiency is determined by the ratio of total sleep time over total time-in-bed.

Sleep quality can be determined by one or more of total sleep time, amount of REM, NREM sleep and Sleep stages, amount of movement and wakefulness and sleep diary, i.e., record of daily sleep hours & the feeling of the next day to know how much sleep necessary for individual.

REM is also sometimes referred to as “dream” sleep. NREM includes 3 stages called N1, N2 and N3.

Many of a user's physiological functions such as brain wave activity, breathing, and heart rate are quite variable during REM sleep, but are extremely regular in NREM sleep.

It has been found that during REM sleep, the brain is restored and captures memories which allow learning to take place, etc. Heart rate, blood pressure and body temperature will typically increase. Generally, 20-25% of total sleep time is REM sleep. N1 is a transition between wakefulness and sleep. N2 is during light sleep, in which the heart rate is slower. Generally, 50-55% of total sleep time is N2 sleep. N3 is during deep sleep to restore the physical body, during which the body temperature and blood pressure will typically decrease.

Sleep Cycle consists of consecutive REM and NREM sleep stages. An average duration for each cycle is about 90 to 110 minutes and there are about 4 to 6 cycles for normal sleeping hours over the night (compare FIG. 1).

There are several devices in the market to monitor sleep efficiency or quality. Polysomnography (PSG) is the current gold standard for sleep study to diagnose sleep disorder. PSG includes the monitoring of many different physiological signals such as heart rate variability (HRV), respiration, electroencephalography (EEG), eletromyography (EMG), electrooculagram (EOG); and it needs to be performed in a sleep laboratory under the supervision of sleep experts. Although PSG is an important tool for sleep diagnosis, it is an uncomfortable and costly procedure, especially when multiple nights of observation are required. Some wearable devices have also been developed to ease these inconveniences. However, those devices are not generally able to measure sleep quality nor sleep efficiency accurately.

Embodiments of the present invention provide at least an alternative device and method for sleep monitoring.

### SUMMARY

According to a first aspect of the present invention there is provided a method for determining time-to-sleep and wake periods during sleep, the method comprising the steps of obtaining motion data representative of motion of a user; detecting the time-to-sleep from the motion data based on a first time-above-threshold (TAT) threshold and a first proportional integration method (PIM) threshold; and detecting the wake periods during sleep from the motion data based on a second TAT threshold and a second PIM threshold.

According to a second aspect of the present invention there is provided a device for determining time-to-sleep and wake periods during sleep, the device comprising a sensor for obtaining motion data representative of motion of a user; and a processor for detecting the time-to-sleep from the motion data based on a first time-above-threshold (TAT) threshold and a first proportional integration device (PIM) threshold; and for detecting the wake periods during sleep from the motion data based on a second TAT threshold and a second PIM threshold.

According to a third aspect of the present invention there is provided a method for determining rapid eye movement (REM) sleep and non REM (NREM) sleep, the method comprising the steps of obtaining physiological signal data of a user; splitting the physiological signal data into respective data subsets; and detecting REM sleep and non REM (NREM) sleep in each data subset based on one or more heart rate variability (HRV) features extracted from each data subset based on adaptive thresholds for each HRV feature.

According to a fourth aspect of the present invention there is provided a device for determining rapid eye movement (REM) sleep and non REM (NREM) sleep, the device comprising a sensor for obtaining physiological signal data of a user; and a processor for splitting the physiological signal data into respective data subsets; and for detecting REM sleep and non REM (NREM) sleep in each data subset based on one or more heart rate variability (HRV) features extracted from each data subset based on adaptive thresholds for each HRV feature.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1 shows a typical sleep profile of a person.

FIG. 2 shows a schematic block diagram of a wearable device according to an example embodiment.

FIG. 3 shows graphs illustrating obtained sleep efficiency, sleep quality, and final sleep stage graphs according to an example embodiment.

FIG. 4 shows a flowchart illustrating a method according to an example embodiment.

FIGS. 5A and 5B show a flowchart and graphs respectively illustrating a detail of the method of FIG. 4 according to an example embodiment.

FIG. 6 shows comparative data of a reference PSG device versus an algorithm using HRV features from a physiological signal according to an example embodiment.

FIG. 7A and FIG. 7B show graphs illustrating a comparison between processing using continuous and on/off detection according to example embodiments respectively.

FIGS. 8A to 8D shows graphs illustrating REM and NREM detection according to an example embodiment.

FIGS. 9A and 9B show graphs illustrating TAT and PIM calculations respectively, according to an example embodiment.

FIG. 10 shows a flowchart illustrating a method according to an example embodiment.

FIG. 11A to 11C show graphs illustrating raw motion data, resultant magnitude data, and TAT and PIM scores respectively, according to an example embodiment.

FIG. 12 shows a graph illustrating sleep-onset determination according to an example embodiment.

FIG. 13 shows a graph illustrating wake-during-sleep determination according to an example embodiment.

FIG. 14 shows a flowchart illustrating usage of a method and device according to an example embodiment.

FIG. 15 shows a schematic diagram illustrating an assembly comprising a wearable device in the form of a wrist watch according to an example embodiment.

FIG. 16 shows a schematic block diagram illustrating an assembly comprising a wearable device according to an example embodiment.

FIG. 17 shows a schematic diagram illustrating a preferred LED-PD configuration for the measurement in reflectance mode for a wearable device of FIG. 15.

FIG. 18 shows a flowchart illustrating a method for determining time-to-sleep and wake periods during sleep according to an example embodiment.

FIG. 19 shows a schematic block diagram illustrating a device for determining time-to-sleep and wake periods during sleep.

FIG. 20 shows a flowchart illustrating a method for determining rapid eye movement (REM) sleep and non REM (NREM) sleep.

FIG. 21 shows a schematic block diagram illustrating a device for determining rapid eye movement (REM) sleep and non REM (NREM) sleep.

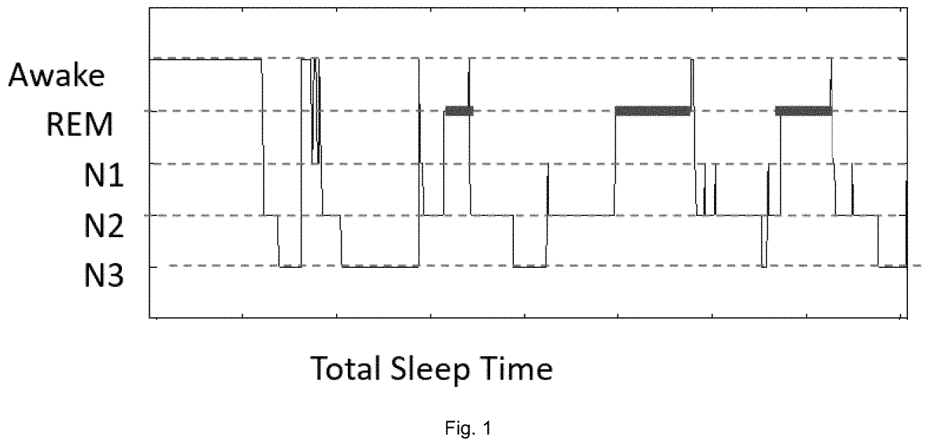

### DETAILED DESCRIPTION

Embodiments of the present invention provide a device and method for sleep monitoring, in particular for determining sleep condition, especially sleep stages (REM, NREM), and/or sleep and wake states.

In the example embodiments described, the sleep stages are determined based on heart rate variability (HRV) and through adaptive thresholds derived from the average of a data-subset to determine sleep cycles. Sleep and wake states are identified based on acceleration magnitude and a combination of TAT (Time-above-threshold) and PIM (Proportional Integration Method) thresholds.

Advantageously, embodiments of the present invention can measure sleep stages accurately and effectively with power consumption efficiency, thus reducing battery spent for the wearable device.

Also, the example embodiments described advantageously provide for accurate detection of sleep-onset latency (time taken to fall asleep) through usage of different threshold levels to further differentiate motions during and before sleep. Stringent TAT and PIM thresholds in each level are applied to differentiate motions related to wakefulness and sleep.

In one embodiment, three stages of wake during sleep, REM sleep, and NREM sleep are calculated simultaneously by using motion data, e.g. an acceleration signal measured by an accelerometer (ACC) sensor or a gyroscope, and physiological signal data, for example a photoplethysmography (PPG) signal measured by a PPG sensor.

Example embodiments use stringent TAT and PIM thresholds obtained from experimental data to differentiate motions related to wakefulness and sleep when both conditions are satisfied. Accurate detection of sleep-onset latency is preferably enabled through the combined usage of high/low threshold levels that are set to further differentiate motions during sleep and when trying to fall asleep. Advantageously, high sensitivity thresholds are sensitive to motions when trying to fall asleep, and low sensitivity thresholds are sensitive to motions during sleeping.

The present specification also discloses an apparatus, which may be internal and/or external to the wearable device in example embodiments, for performing the operations of the methods. Such apparatus may be specially constructed for the required purposes, or may comprise a general purpose computer or other device selectively activated or reconfigured by a computer program stored in the computer. The algorithms and displays presented herein are not inherently related to any particular computer or other apparatus. Various general purpose machines may be used with programs in accordance with the teachings herein. Alternatively, the construction of more specialized apparatus to perform the required method steps may be appropriate. The structure of a conventional general purpose computer will appear from the description below. In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a general purpose computer. The computer readable medium may also include a hard-wired medium such as exemplified in the Internet system, or wireless medium such as exemplified in the GSM mobile telephone system. The computer program when loaded and executed on such a general-purpose computer effectively results in an apparatus that implements the steps of the preferred method.

The invention may also be implemented as hardware modules. More particularly, in the hardware sense, a module is a functional hardware unit designed for use with other components or modules. For example, a module may be implemented using discrete electronic components, or it can form a portion of an entire electronic circuit such as an Application Specific Integrated Circuit (ASIC). Numerous other possibilities exist. Those skilled in the art will appreciate that the system can also be implemented as a combination of hardware and software modules.

The described embodiments of the invention described herein relate to a wearable device and a method for sleep monitoring based on motion signals acquired from the user with a motion sensor such as an ACC and/or a gyroscope and based on physiological signals acquired from the user with sensor such as PPG sensor.

In one embodiment, the device can be worn on any location of the user with sufficient skin area to allow the light emitting diode-photo detector (LED-PD) arrangement to acquire the PPG signal and allows the tri-axial ACC to acquire motion signals.

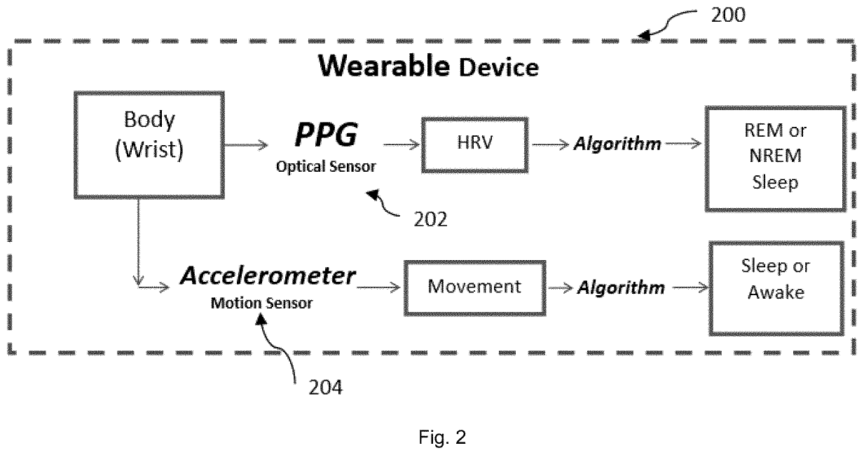

The device 200 according to an example embodiment shown in FIG. 2 is in the form of a wrist actigraphy with accelerometer and PPG sensor. The device 200 measures heart rate variability (HRV) from the PPG signal measured by a PPG sensor 202 and detects REM/NREM sleep. The accelerometer 204 detects movement and measures sleep and awake-during-sleep time, Sleep-onset latency (time taken to fall asleep), and Sleep efficiency (Total sleep time/Total time in bed).

### Overall Sleep Assessment in an Example Embodiment

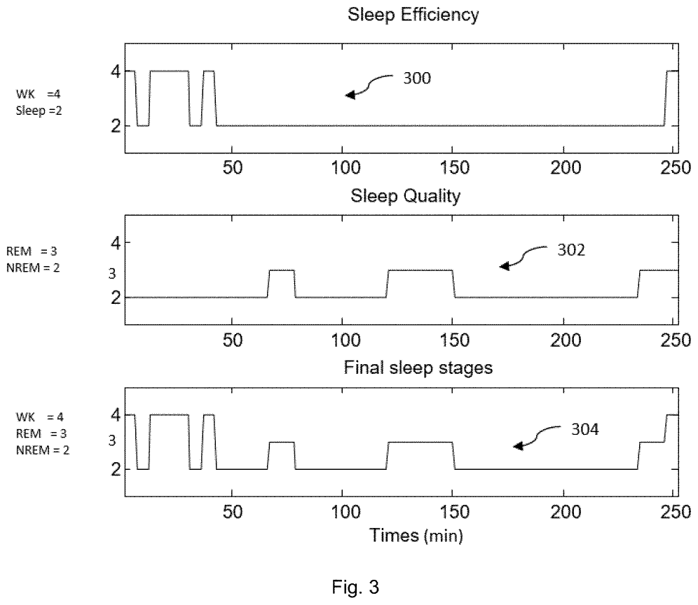

With reference to FIG. 3, determination of wake or sleep (curve 300), and determination of REM or NREM (curve 302) are conducted simultaneously in an example embodiment, and both results are combined to provide a final result (curve 304) of wake, REM sleep and NREM sleep time.

### Classification of REM and NREM Sleep in an Example Embodiment

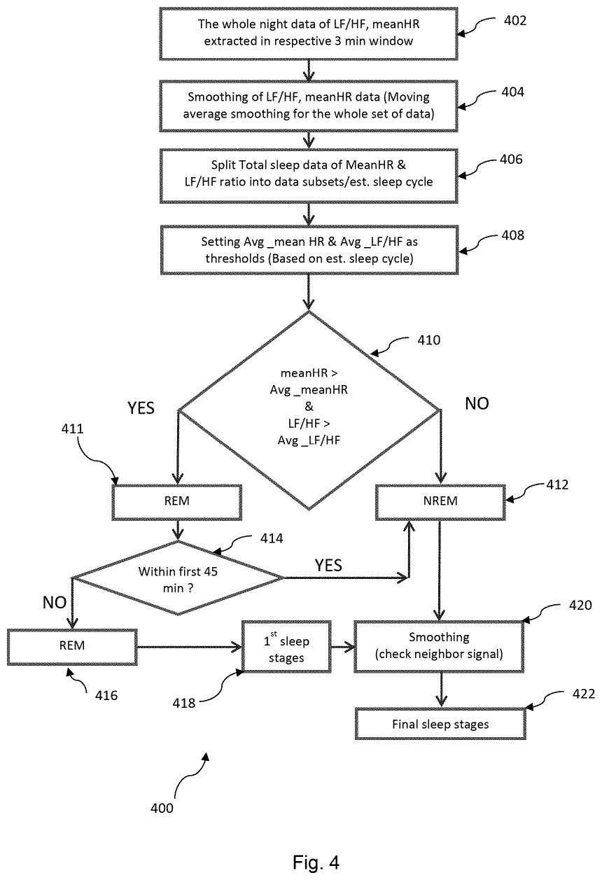

FIG. 4 shows a flow chart 400 illustrating classification of REM and NREM sleep in an example embodiment. HRV features in the frequency domain and time domain are extracted from PPG signal in 3 min duration, in an on/off operation mode. More particular, a whole night of data for low frequency/high frequency (LF/HF) ratio and mean heart rate (meanHR) are extracted from the PPG signal in respective 3 min duration (step 402). For example, the LF range may be from about 0.04 to 0.15 Hz, and the HF range may be from about 0.15 to 0.4 Hz. As is understood in the art, LF/HF decreases in NREM sleep due to greater parasympathetic modulation and increases in REM sleep due to greater sympathetic modulation. Mean HR, which is representative of the variation of the heart rate, decreases or stables in NREM sleep and increases and varies in REM sleep. Optionally, smoothing of the LF/HF data and the mean HR data is performed (step 404), e.g. moving average smoothing for the whole data set for one night.

The total sleep data is split into subsets corresponding to each estimated sleep cycle duration (step 406), and thresholds are set (step 408). For example, a sleep cycle is estimated as 1 hr. Thresholds are set based on the average during each subset/estimated sleep cycle, in the example embodiment.

REM sleep is determined when the HRV features are greater than the thresholds, otherwise determined as NREM sleep (step 410-412). If REM sleep is determined for data falling within the initial period of the whole night data (step 414), for example within the first 45 minutes, the determination is changed to NREM (step 412), otherwise the REM determination is maintained (step 416). The combination of the REM sleep and NREM sleep determinations is used to generate a first or intermediate result for the sleep stages (step 418), where S(i) indicates the sleep stage result in each 3 min measurement interval in the example embodiment. For example, S(i)=3 for REM stage result, and S(i)=2 for NREM stage result.

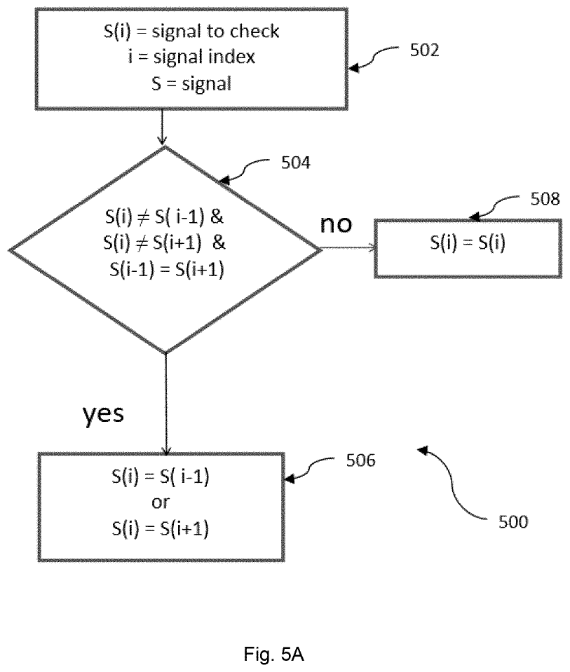

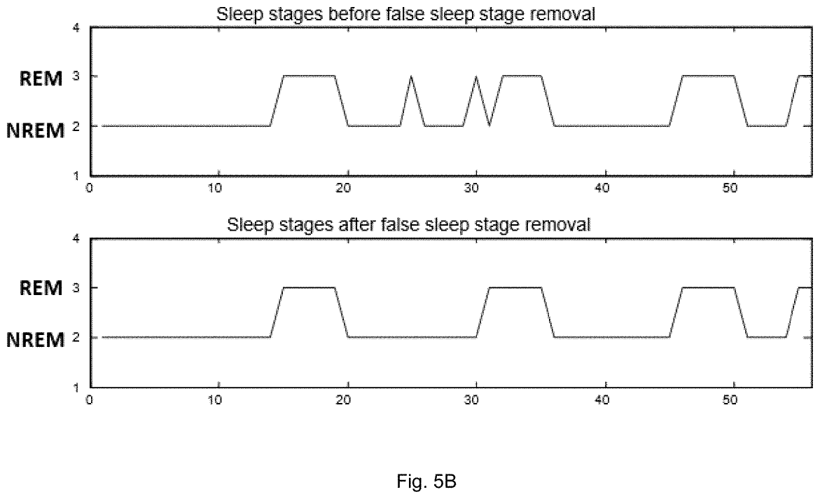

Smoothing by checking nearest neighbor of sleep stage results for a 3 min measurement interval (step 420) is performed, to remove false condition prior to output of the final sleep stages result (step 422). Details of the nearest neighbor checking method in the example embodiment are shown in the flowchart 500 in FIG. 5a). At step 502, S(i) is the sleep stage result for a 3 min measurement interval to be checked. At step 504, it is determined whether S(i) is not the same as S(i−1), whether S(i) is not the same as S(i+1), and whether S(i−1) is the same as S(i+1). If all conditions are fulfilled, S(i) is replaced with S(i−1) or S(i+1) (noting S(i−1)=S(i+1) if conditions are fulfilled), see step 506. Otherwise, S(i) is maintained, i.e. S(i)=S(i), see step 508. FIG. 5b) shows graphs 510, 512 illustrating the sleep stages results before and after false sleep stage removal according to the example embodiment.

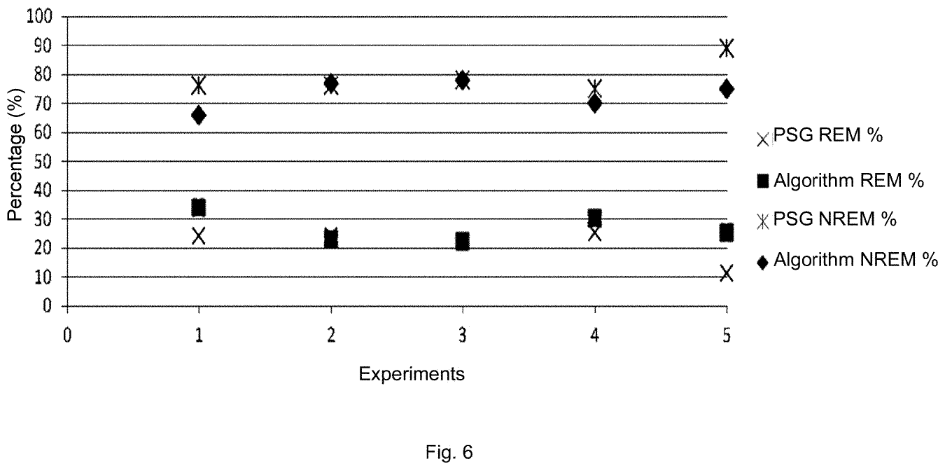

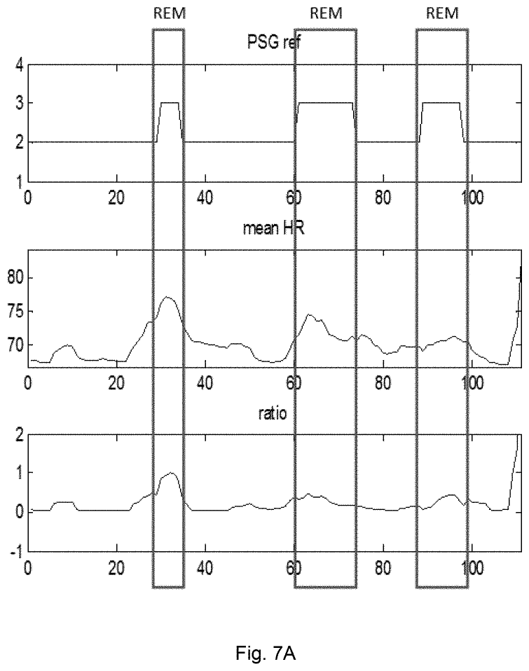

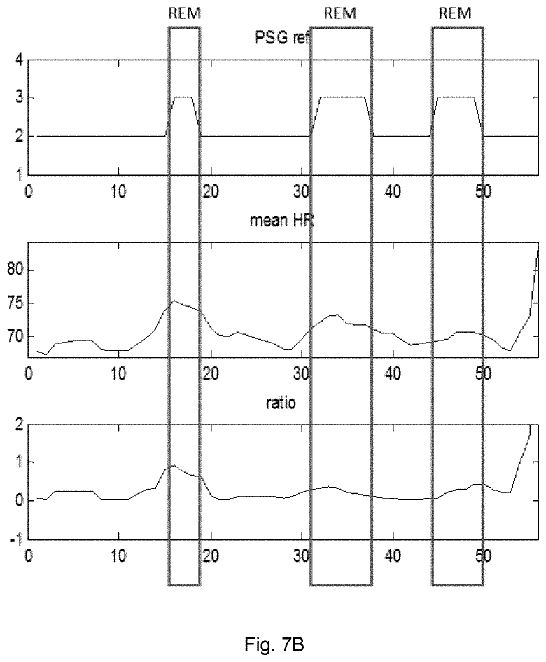

FIG. 6 shows comparative data of a reference PSG device (“PSG REM %” and “PSG NREM %”) versus an algorithm using HRV features from a physiological signal according to an example embodiment (“Algorithm REM %” and “Algorithm NREM %). As mentioned above, the HRV features are extracted from the PPG signal in 3 min ON/OFF duration in an example embodiment. Continuous monitoring may be considered as ideal but it consumes battery. The inventors unexpectedly found that measuring in ON/OFF duration, for example in 3 min ON/OFF duration can provide similar result compared to continuous monitoring. FIGS. 7a) and b) show results based on continuous monitoring (i.e. 135 windows of 3 min duration each) and based on ON/OFF duration (here 68 windows of 3 min each over the same total time period) respectively. Accordingly, power consumption can advantageously be reduced for a wearable device according to an example embodiment while maintaining an acceptable accuracy.

### Sleep Cycle Results According to an Example Embodiment

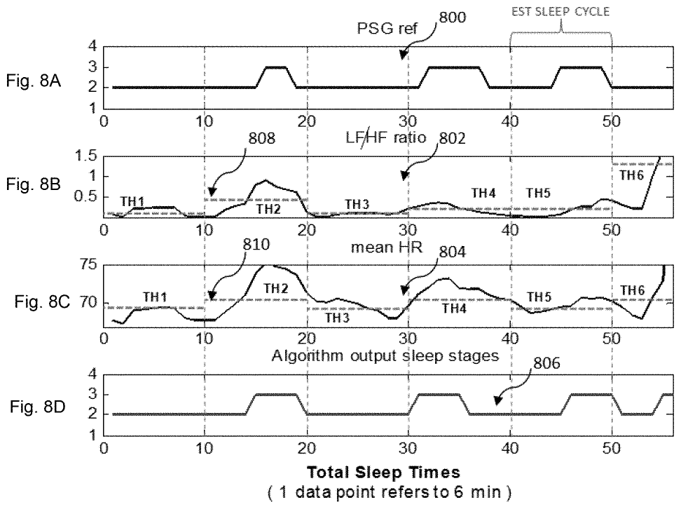

The Sleep cycle is estimated as 1 hr in the example embodiment, and experimental results show a close relation compared to PSG reference. By estimating the sleep cycle as 1 hr, we made the calculation process simple and effective. FIGS. 8a) to d) show graphs showing PSG reference data (curve 800), LF/HF ratio measurement data according to an example embodiment (curve 802), mean HR measurement data according to the example embodiment (curve 804), and the algorithm output of the sleep stages according to the example embodiment (curve 806) respectively. In FIGS. 8b) and c), the adaptive thresholds e.g. 808, 810, for each estimated sleep cycle subset are also shown.

### Sleep Wake Assessment According to an Example Embodiment

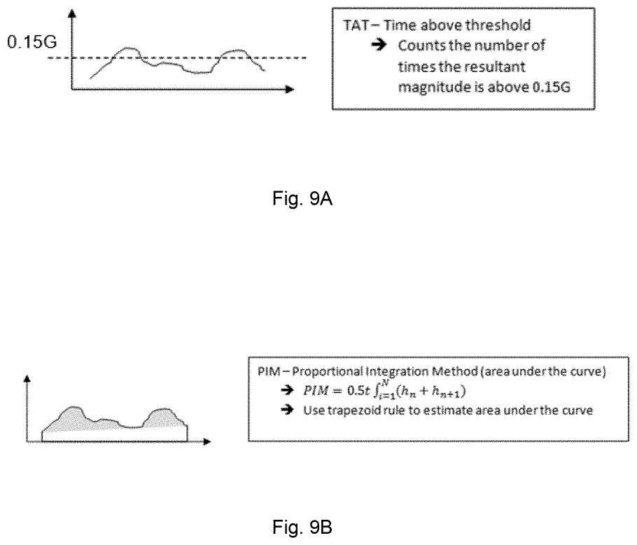

As illustrated in FIG. 9a), TAT (Time-above-threshold) in the example embodiment counts the number of times when the acceleration amplitude is above a set threshold (set at about 0.15 G in one example, set in a range from about 0.1-0.2 G in different embodiments), i.e. TAT reflects the duration and frequency of movements. As illustrated in FIG. 9b), PIM (Proportional Integration Method) integrates the acceleration magnitude signal and calculates the area under the curve using the equation shown in FIG. 9b) in the example embodiment.

By using both TAT and PIM, the results the example embodiment advantageously reflect substantially all the important factors of movement, including duration, frequency, acceleration and amplitude.

On the other hand, the inventors have unexpectedly found that the ZCM (Zero Crossing Mode) parameter, which is often used in existing techniques, does not fully describe the motion and provides less information related to jerk or toss movement. This is illustrated in Table 2 below.

TABLE 2

| Motions | TAT Score | PIM Score | ZCM Score |
|---|---|---|---|
| Jerk (1x) | 0 | 12 | 5 |
| Jerk (3x) | 0 | 17 | 16 |
| Quick Toss (1x) | 147 | 100 | 3 |
| Quick Toss (2x) | 417 | 226 | 2 |
| Slow Toss (1x) | 118 | 74 | 3 |
| Slow Toss (2x) | 346 | 158 | 6 |

Large movements (i.e. toss) during sleep are assumed very uncommon during light and deep sleep due to the body slowing down for physical restoration. It is however possible to have sudden muscle jerks, but these are unrelated to wakefulness.

In the example embodiment, lower sensitivity level thresholds for detecting wake-during-sleep are set to 90% of the “Slow Toss (1×)” during sleep values for the TAT and PIM scores, noting again that the example embodiment does deliberately not use ZCM scores for the reasons explained above and illustrated in Table 2.

For very small movements (i.e. jerk), the values of TAT and PIM are very low. In the example embodiment, the thresholds are set at a higher sensitivity level based on the values for “Jerk (1×)” to identify small movements. Because small movements are unlikely related to movements made when awake, these higher sensitivity level thresholds are used in the example embodiment to identify time-to-sleep.

As mentioned above, for larger movements (i.e. toss), the values of TAT and PIM are much higher. The thresholds are set at this lower sensitivity level to identify larger movements that are better correlated to restlessness/wakefulness during sleep in the example embodiments to identify wakefulness/restlessness during sleep, also referred to herein as wake period during sleep, or wake-during-sleep.

In one embodiment, the higher sensitivity threshold levels for TAT & PIM are set to be 1 and 10 respectively, and the lower sensitivity threshold levels for TAT & PIM are set to be 100 and 62 respectively. It is noted again that to identify wake-during-sleep status and Time-to-sleep, both criteria derived from TAT and PIT score need to be satisfied in the example embodiment, to advantageously make the result more accurate. No ZCM scores are used in this example embodiment.

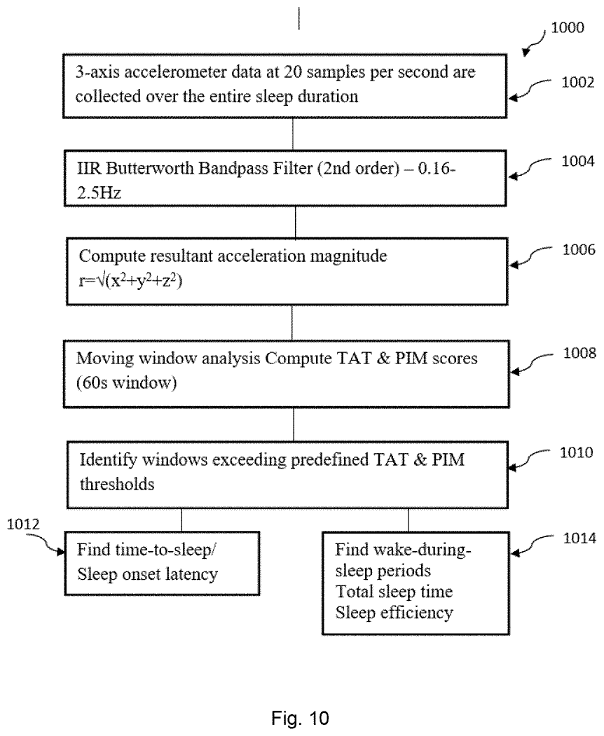

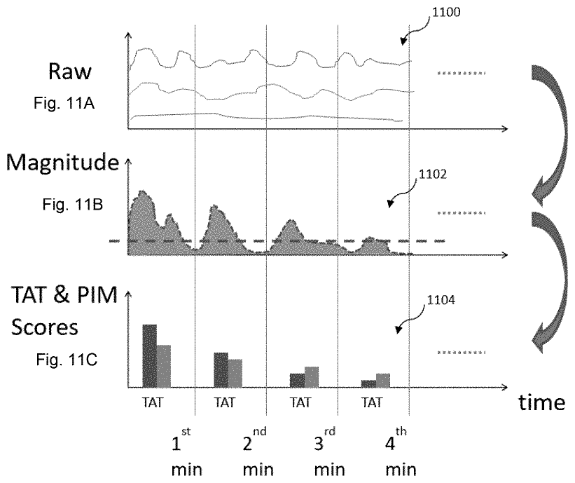

FIG. 10 shows a flow chart 1000 illustrating the wake-during-sleep status and Time-to-sleep determination algorithm according to an example embodiment. FIGS. 11a) to c) show graphs illustrating the raw 3-axis motion data obtained in an example embodiment (graph 1100), the resultant magnitude signal calculated (curve 1102), and the TAM and PIM scores in respective 1 minute periods (graph 1104).

Returning to FIG. 10, acceleration magnitude data is collected from the wrist-worn 3-axis accelerometer at 20 samples per second for the whole sleep duration (step 1002). After bandpass filtering (step 1004), the resultant of the 3 axis acceleration magnitude is calculated by RMS (step 1006). The frequency range of interest in the example embodiment is between about 0.16 to 2.5 Hz. The acceleration magnitude is processed every 60 seconds to derive TAT and PIM actigraph scores (step 1008).

Six sleep parameters can be calculated in an example embodiment. The six parameters are Time-to-sleep, Number of awakenings, Total time awake during actual sleep period, Total sleep time, Total time-in-bed, and Sleep efficiency.

Time-to-sleep (sleep-onset latency) is identified based on high sensitivity thresholds (steps 1010 and 1012). If both TAT and PIM scores are lower than the high sensitivity thresholds, the 60 seconds window is classified as quiet period, and quiet period must satisfy consecutive ‘N’ windows, i.e. N windows of little or no movements. N can be about 5-20, preferably about 8-15 in example embodiments. Wake periods during sleep are identified when TAT and PIM scores exceed pre-determined low sensitivity thresholds (steps 1010 and 1014). If both TAT and PIM are higher than the low sensitivity thresholds, the 60 seconds window is classified as wake period during sleep.

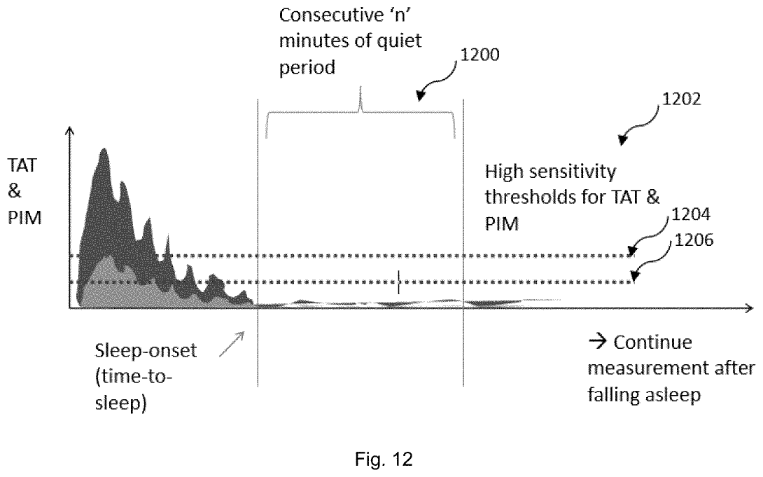

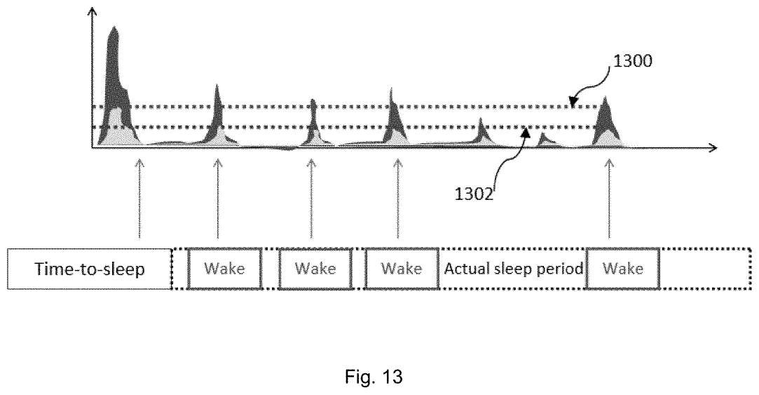

FIG. 12 shows TAT and PIM scores measured according to an example embodiment, illustrating the consecutive N windows (minutes in the example embodiment) 1200, determined based on the low sensitivity thresholds, and application of the high sensitivity thresholds thereafter (indicated at numeral 1202), i.e. after falling asleep. The horizontal lines 1204, 1206 show the low sensitivity thresholds for TAT and PIM respectively. FIG. 13 shows the TAT scores measured over an extended period according to an example embodiment, noting that the horizontal lines 1300, 1302 show the high sensitivity thresholds for TAT and PIM respectively.

Sleep Efficiency is determined by calculating Total sleep time/Total time in bed. Actionable feedback can be provided for sleep efficiency, MSLT score, Sleep debt and optimal alarm function. If sleep efficiency is greater than about 85%, it can be considered normal according to current understanding. The MSLT score can be used to show how serious the user's sleep deprivation is. Sleep debt shows whether the user is getting enough sleep hours. Optimal alarm function can be set and vibration can be used in an example embodiment.

### Usage Flowchart According to an Example Embodiment

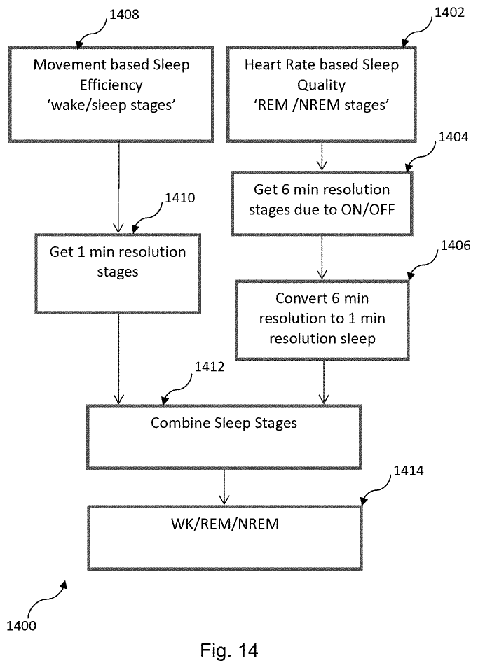

FIG. 14 shows a flowchart (1400) illustrating usage of the device and method according to an example embodiment. HRV features (meanHR and LF/HF ratio) for sleep quality are calculated in real time from the physiological signal sensor data for the whole night. The data processing for sleep stages (REM/NREM), indicated at step 1402, step 1404 (get 6 min resolution stages due to on/off) and step 1406 (convert 6 min resolution to 1 min resolution sleep stages), start once the user exits the sleep mode. Sleep efficiency data (i.e. determine wake during sleep/sleep stages) are calculated in real time from the motion sensor data, indicated at step 1408. The data processing for getting 1 min resolution stages at step 1410 starts once the user exits the sleep mode. The results are combined at step 1412, for output of the final sleep stages results at step 1414.

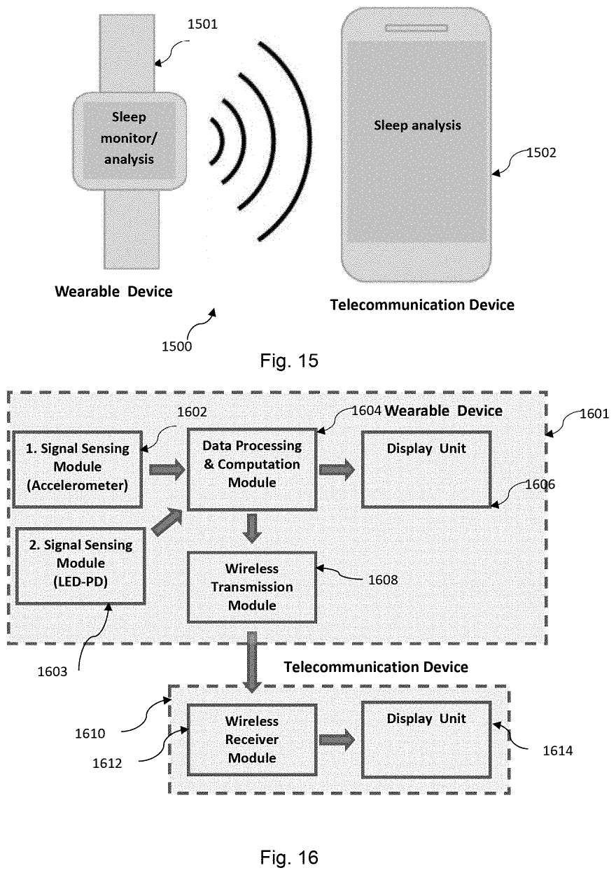

FIG. 15 shows an assembly 1500 comprising a wearable device in the form of a wrist watch 1501 according to an example embodiment. It will be appreciated that in different embodiments the device may also be in any other form suitable to be worn on any part of the user's body such as his/her arms, waist, hip or foot. The wrist watch 1501 obtains physiological measurements and motion data from a user, processes the data, presents result(s) and communicates the result(s) wirelessly to a telecommunication device of the assembly 1500 such as a mobile phone 1502 or other portable electronic devices, or computing devices such as desk top computers, laptop computer, tab computers etc.

FIG. 16 shows a schematic block diagram of an assembly 1600 comprising a wearable device 1601 according to an example embodiment, for obtaining physiological measurements from a user and removing artifacts in the physiological measurements. The device 1601 includes a first signal sensing module 1602, such as an accelerometer or gyroscope, for obtaining the motion information of the user.

One non-limiting example of a preferred accelerometer that can be adapted for use in the device is a triple-axis accelerometer MMA8652FC available from Freescale Semiconductor, Inc. This accelerometer can provide the advantage of measuring acceleration in all three directions with a single package. Alternatively, several single-axis accelerometers oriented to provide three-axis sensing can be used in different embodiments.

The device 1601 also includes a second sensing module 1603, such as an LED-PD module, for obtaining a physiological signal of the user. The device 1601 also includes a data processing and computational module 1604, such as a processor, which is arranged to receive and process the acceleration information from the signal sensing module 1602 and the physiological signal from the measurement module 1603. The device 1601 also includes a display unit 1606 for displaying a result to a user of the device 1601 and for receiving user input via touch screen technology. The device 1601 in this embodiment further includes a wireless transmission module 1608 arranged to communicate wirelessly with a telecommunications device 1610 of the assembly 1600. The telecommunication device 1610 includes a wireless receiver module 1612 for receiving signals from the wearable device 1601, a display unit 1614 for displaying a result to a user of the telecommunication device 1610 and for receiving user input via touch screen technology.

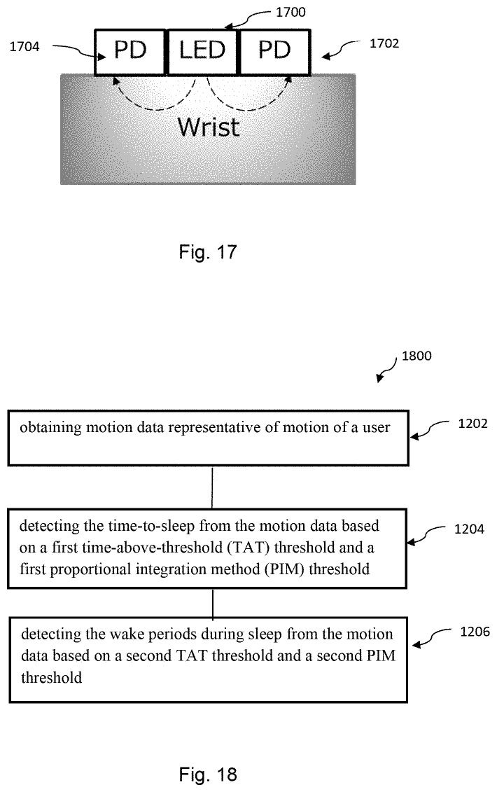

FIG. 17 shows a schematic illustration of preferred LED-PD configuration for the measurement in reflectance mode for a wearable device in the form of wrist watch 1701. The measurement is based on the amount of light by a LED 1700 reflected back to two PDs 1702, 1704. One non-limiting example of a preferred LED-PD module that can be adapted for use in the device is composed of one LED, e.g. OneWhite Surface Mount PLCC-2 LED Indicator ASMT-UWB1-NX302, paired with one or multiple PDs, e.g. ambient light sensor TEMD5510FX01. Alternatively, the LED-PD module can be composed of multiple LEDs paired with one or multiple PDs.

FIG. 18 shows a flowchart 1800 illustrating a method for determining time-to-sleep and wake periods during sleep according to an example embodiment. At step 1802, motion data representative of motion of a user is obtained. At step 1804, the time-to-sleep is detected from the motion data based on a first time-above-threshold (TAT) threshold and a first proportional integration method (PIM) threshold. At step 1806, the wake periods during sleep are detected from the motion data based on a second TAT threshold and a second PIM threshold.

The first and second TAT thresholds may be different. The first TAT threshold may be lower than the second TAT threshold.

The first and second PIM thresholds may be different. The first PIM threshold may be lower than the second PIM threshold.

Detecting the time-to-sleep from the motion data may comprise dividing the motion data into time windows; determining TAT and PIM scores for each time window, and identifying windows in which the TAT and PIM scores are below the first TAT threshold and the first PIM threshold.

Detecting the wake periods during sleep from the motion data may comprise dividing the motion data into time windows; determining TAT and PIM scores for each time window, and identifying windows in which the TAT and PIM scores exceed the second TAT threshold and the second PIM threshold.

The motion data may comprise multi-axis motion signals. The method may further comprise calculating a resultant magnitude of the multi-axis motion signals using bandpass filtering and root-mean-square (RMS) calculation.

The first and second TAT thresholds may be respective number of times a magnitude derived from the motion data is above an acceleration threshold. The acceleration threshold may be in a range from 0.1 to 0.2 G, and preferably about 0.15 G. The first and second PIM thresholds may be respective areas under a magnitude curve derived from the motion data. The respective areas may be estimated using a trapezoid rule.

The determining of the time-to-sleep and wake periods during sleep may not be based on zero-crossing-mode detection based on the motion data.

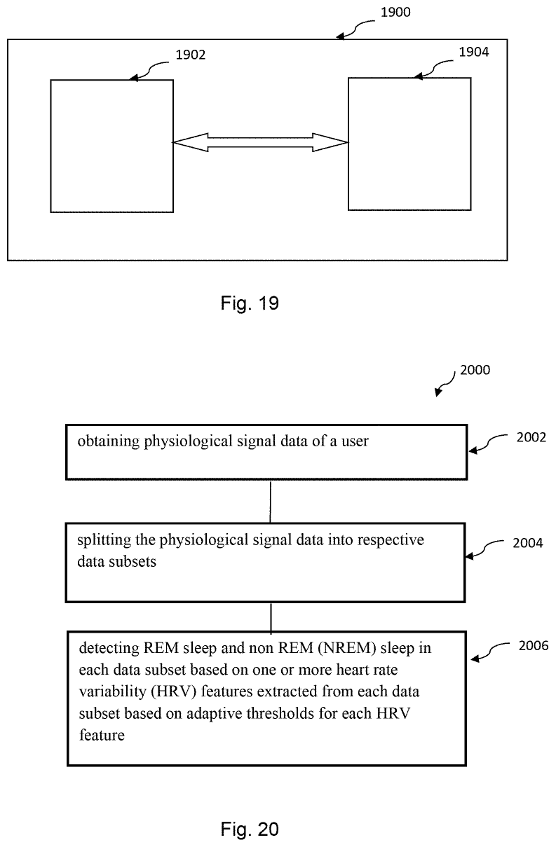

FIG. 19 shows a schematic block diagram illustrating a device 1900 for determining time-to-sleep and wake periods during sleep. The device 1900 comprises a sensor 1902 for obtaining motion data representative of motion of a user; and a processor 1904 for detecting the time-to-sleep from the motion data based on a first time-above-threshold (TAT) threshold and a first proportional integration device (PIM) threshold; and for detecting the wake periods during sleep from the motion data based on a second TAT threshold and a second PIM threshold.

The first and second TAT thresholds may be different. The first TAT threshold may be lower than the second TAT threshold.

The first and second PIM thresholds may be different. The first PIM threshold may be lower than the second PIM threshold.

Detecting the time-to-sleep from the motion data may comprise dividing the motion data into time windows; determining TAT and PIM scores for each time window, and identifying windows in which the TAT and PIM scores are below the first TAT threshold and the first PIM threshold.

Detecting the wake periods during sleep from the motion data may comprise dividing the motion data into time windows; determining TAT and PIM scores for each time window, and identifying windows in which the TAT and PIM scores exceed the second TAT threshold and the second PIM threshold.

The motion data may comprise multi-axis motion signals. The processor may further be configured for calculating a resultant magnitude of the multi-axis motion signals using bandpass filtering and root-mean-square (RMS) calculation.

The first and second TAT thresholds may be respective number of times a magnitude derived from the motion data is above an acceleration threshold. The acceleration threshold may be in a range from 0.1 to 0.2 G, and preferably about 0.15 G.

The first and second PIM thresholds may be respective areas under a magnitude curve derived from the motion data. The respective areas may be estimated using a trapezoid rule.

The determining of the time-to-sleep and wake periods during sleep may not be based on zero-crossing-mode detection based on the motion data.

FIG. 20 shows a flowchart 2000 illustrating a method for determining rapid eye movement (REM) sleep and non REM (NREM) sleep. At step 2002, physiological signal data of a user is obtained. At step 2004, the physiological signal data is split into respective data subsets. At step 2006, REM sleep and non REM (NREM) sleep are detected in each data subset based on one or more heart rate variability (HRV) features extracted from each data subset based on adaptive thresholds for each HRV feature.

Detecting REM sleep and NREM sleep may comprise detecting REM sleep in respective time windows within the data subset. The time windows may correspond to on-stages of a detector for the physiological signal data, the detector operating in an on/off operation mode. The on-stage may be about 3 minutes and the detector may operate in an about 50% on/off operation mode.

In each time window REM sleep and NREM may be detected based on the adaptive thresholds.

The method may further comprise changing a detected REM sleep to a detected NREM sleep if the detected REM sleep is within an initial time period of the obtained physiological signal data. The initial time period may be about 45 minutes.

The method may further comprise comparing a REM sleep and NREM sleep detection result for one time window with respective results for its nearest neighboring time windows. The method may comprise maintaining the detection result in said one window if said detection result is similar to the respective results for the nearest neighboring time windows, and changing said detection result otherwise.

The HRV features may comprise a mean heart rate (meanHR) and a low frequency/high frequency (LF/HF) ratio derived from the physiological signal data.

A first adaptive threshold may be an average of a first HRV feature in each data subset. A second adaptive threshold may be an average of a second HRV feature in each data subset. REM sleep may be detected if the first HRV feature is above a first adaptive threshold and the second HRV feature is above a second adaptive threshold, and NREM sleep may be detected otherwise.

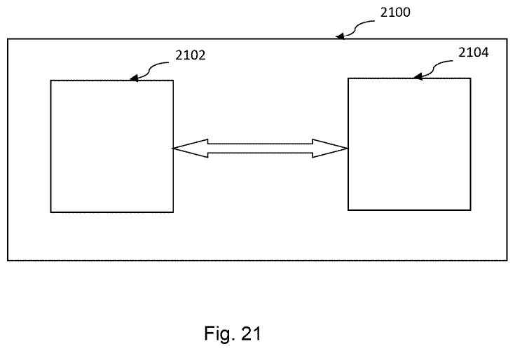

FIG. 21 shows a schematic block diagram illustrating a device 2100 for determining rapid eye movement (REM) sleep and non REM (NREM) sleep. The device 2100 comprises a sensor 2102 for obtaining physiological signal data of a user, and a processor 2104 for splitting the physiological signal data into respective data subsets; and for detecting REM sleep and non REM (NREM) sleep in each data subset based on one or more heart rate variability (HRV) features extracted from each data subset based on adaptive thresholds for each HRV feature.

Detecting REM sleep and NREM sleep may comprise detecting REM sleep in respective time windows within the data subset. The time windows may correspond to on-stages of a detector for the physiological signal data, the detector operating in an on/off operation mode. The on-stage may be about 3 minutes and the detector may operate in an about 50% on/off operation mode.

In each time window REM sleep and NREM may be detected based on the adaptive thresholds.

The processor 2104 may further be configured for changing a detected REM sleep to a detected NREM sleep if the detected REM sleep is within an initial time period of the obtained physiological signal data. The initial time period may be about 45 minutes. The processor 2104 may further be configured for comparing a REM sleep and NREM sleep detection result for one time window with respective results for its nearest neighboring time windows. The processor 2104 may be configured for maintaining the detection result in said one window if said detection result is similar to the respective results for the nearest neighboring time windows, and for changing said detection result otherwise.

The HRV features may comprise a mean heart rate (meanHR) and a low frequency/high frequency (LF/HF) ratio derived from the physiological signal data.

A first adaptive threshold may be an average of a first HRV feature in each data subset. A second adaptive threshold may be an average of a second HRV feature in each data subset. REM sleep may be detected if the first HRV feature is above a first adaptive threshold and the second HRV feature is above a second adaptive threshold, and NREM sleep may be detected otherwise.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive. Also, the invention includes any combination of features, in particular any combination of features in the patent claims, even if the feature or combination of features is not explicitly specified in the patent claims or the present embodiments.

For example, while a wrist-worn device is described in some embodiments, the device may be worn on any part of the arms, hip, waist or foot of the user.

Also, according to the human sleep behavior as currently understood, reduction in heart rate and blood pressure occur during NREM sleep. In REM sleep, there is more variation in cardiovascular activity which can cause overall increases in blood pressure and heart rate. The example embodiments described employ mean HR and LF/HF as HRV features. However, it will be appreciated that different HRV features (e.g Standard Deviation of Heart Rate for period of interest (SDHR), Percent of NN intervals>50 ms different from previous (NN) for period of interest (PNN50), Root mean square of successive differences of NN interval for period of interest (RMSSD) and blood flow features (e.g mean Pulse Pressure for the period of interest (mean PP), Average standard deviation of pulse pressure for the period of interest (ASDPP)) can additionally or alternatively be applied in different embodiments, to improve performance.

What is claimed is:

1. A method for determining rapid eye movement (REM) sleep and non REM (NREM) sleep, the method comprising:
obtaining physiological signal data of a user;
splitting the physiological signal data into respective data subsets; and
detecting REM sleep and non REM (NREM) sleep in each data subset based on one or more heart rate variability (HRV) features extracted from each data subset based on at least one threshold for each HRV feature.

2. The method as claimed in claim 1, wherein detecting REM sleep and NREM sleep comprises detecting REM sleep in respective time windows within the data subset.

3. The method as claimed in claim 2, wherein in each time window REM sleep and NREM are detected based on the at least one threshold.

4. The method as claimed in claim 1, further comprising changing a detected REM sleep to a detected NREM sleep if the detected REM sleep is within an initial time period of the obtained physiological signal data.

5. The method as claimed in claim 4, wherein the initial time period is within about 45 minutes.

6. The method as claimed in claim 2, further comprising comparing a REM sleep and NREM sleep detection result for one time window with respective results for its nearest neighboring time windows.

7. The method as claimed in claim 6, comprising maintaining the detection result in said one window if said detection result is similar to the respective results for the nearest neighboring time windows, and changing said detection result otherwise.

8. The method as claimed in claim 1, wherein the HRV features comprise one or more of a group consisting of a mean heart rate (meanHR), average standard deviation of pulse pressure (ASDPP), standard deviation of heart rate for period of interest (SDHR), percent of NN intervals>50 ms different from previous NN for period of interest (PNN50), root mean square of successive differences of NN interval for period of interest (RMSSD), blood flow features, and mean pulse pressure for the period of interest (mean PP), derived from the physiological signal data.

9. A device for determining rapid eye movement (REM) sleep and non REM (NREM) sleep, the device comprising:
a sensor for obtaining physiological signal data of a user; and
a processor for splitting the physiological signal data into respective data subsets; and for detecting REM sleep and non REM (NREM) sleep in each data subset based on one or more heart rate variability (HRV) features extracted from each data subset based on at least one threshold for each HRV feature.

10. The device as claimed in claim 9, wherein detecting REM sleep and NREM sleep comprises detecting REM sleep in respective time windows within the data subset.

11. The device as claimed in claim 10, wherein in each time window REM sleep and NREM are detected based on the at least one threshold.

12. The device as claimed in claim 9, further comprising changing a detected REM sleep to a detected NREM sleep if the detected REM sleep is within an initial time period of the obtained physiological signal data.

13. The device as claimed in claim 12, wherein the initial time period is within about 45 minutes.

14. The device as claimed in claim 9, wherein the processor is further configured for comparing a REM sleep and NREM sleep detection result for one time window with respective results for its nearest neighboring time windows.

15. The device as claimed in claim 14, comprising wherein the processor is configured for maintaining the detection result in said one window if said detection result is similar to the respective results for the nearest neighboring time windows, and for changing said detection result otherwise.

16. The device as claimed in claim 9, wherein the HRV features comprise one or more of a group consisting of a mean heart rate (meanHR), an average standard deviation of pulse pressure (ASDPP), standard deviation of heart rate for period of interest (SDHR), percent of NN intervals>50 ms different from previous NN for period of interest (PNN50), root mean square of successive differences of NN interval for period of interest (RMSSD), blood flow features, and mean pulse pressure for the period of interest (mean PP), derived from the physiological signal data.
